(19) United States
(12) Patent Application Publication
(10) Pub. No.: US 2002/0160445 A1
(43) Pub. Date: Oct. 31, 2002

(54) Method of providing polypeptide preparations with reduced enzymatic side activities

(76) Inventor: Marianne Harboe, Lyngby (DK)

Correspondence Address: Stanislaus Aksman, Hunton & Williams, Suite 1200, 1900 K Street, N.W., Washington, DC, 20006, US

(21) Appl. No.: 09/779,560

(22) Filed: Feb. 9, 2001

(51) Int. Cl. C12N 9/99; C12P 21/02

(52) U.S. Cl. 435/69.1; 435/184

(57) ABSTRACT

A method of providing polypeptide preparations having a reduced content of undesired enzymatic side activities, the method comprising subjecting a medium containing a desired polypeptide such as an enzyme, a pharmaceutically active or immunologically active polypeptide to a pH of less than 2 for a period of time that is required to inactivate the side activities whilst retaining the activity of the desired polypeptide. The method is useful for providing milk clotting enzyme products including rennets or coagulants based on chymosin or pepsin or microbial aspartic proteases e.g. derived from bacterial species and species of filamentous fungi.

### FIELD OF INVENTION

The present invention relates generally to processes of obtaining preparations of polypeptides having a low content of undesired enzymatic activities (side activities). In particular, the invention provides a simple and convenient process whereby the level of such side activities can be reduced significantly or substantially completely inactivated in crude and more or less purified polypeptide preparations such as preparations of aspartic proteases including chymosin species and microbial aspartic proteases.

### TECHNICAL BACKGROUND AND PRIOR ART

A large range of polypeptide products including enzymes and pharmaceutically active products are currently manufactured and made commercially available. Such products may be derived from a variety of sources. Thus, they can be derived from extracts of plant, animal or microbial cells naturally producing the products or they can be manufactured using appropriate recombinant microbial host organisms producing the desired product(s) either being accumulated intracellularly or excreted into the cultivation medium. In any case, the primary crude extract or medium resulting from the cultivation of the recombinant organisms normally contain, in addition to the desired product(s), a range of undesired enzymatic activities naturally produced by the source organisms or the recombinant host cells. In certain instances, an undesired enzymatic activity in a cultivation medium for a recombinant organism is derived from the fact that the desired product is produced as a fusion protein of the desired gene product and a fusion partner having, in relation to the final product, an undesired enzymatic side activity.

It is therefore a continuing concern for the industry how to provide polypeptide products that do not contain undesired enzymatic side activities or at least have a content of such activities at such a low level that they do not restrict the applicability of the polypeptide products for their intended purposes.

One illustrative example of such a problem is the that facing manufacturers of milk clotting enzymes, also referred to as rennets or coagulants. Milk clotting enzymes, which belong to the class of aspartic proteases are widely used in the cheese manufacturing industry to provide a curd of the major milk proteins, the caseins. Commercially available milk clotting enzymes include native enzymes derived from microbial species or animal tissue sources such as calf stomachs, or such enzymes can be provided as gene products of recombinant cells expressing a heterologous milk clotting enzyme of animal or microbial origin.

The industrially most important milk clotting enzymes of animal origin are chymosin and pepsin. When produced in the animal gastric mucosal cells, chymosin and pepsin occur as enzymatically inactive pre-prochymosin and pre-pepsinogen, respectively. When chymosin is excreted, an N-terminal peptide fragment, the pre-fragment (signal peptide) is cleaved off to give prochymosin including a pro-fragment. Prochymosin is a substantially inactive form of the enzyme which, however, becomes activated under acidic conditions to the active chymosin by autocatalytic removal of the pro-fragment This activation occurs in vivo in the gastric lumen under appropriate pH conditions or in vitro under acidic conditions.

Traditionally, milk clotting enzymes of bovine origin have been marketed in the form of extracts of stomach tissues. However, bovine chymosin is increasingly being manufactured industrially using recombinant DNA technology, e.g. using filamentous fungi such as Aspergillus species (see e.g. Ward, 1990), yeast strains, e.g. of Klyuveromyces species, or bacterial species, e.g. E. coli, as host organisms.

Milk clotting enzymes of microbial origin are currently in commercial use in the dairy industry. In the following, such enzymes are also referred to as microbial clotting enzymes, microbial rennets or microbial coagulants. Examples of such enzymes include aspartic proteases natively produced by the filamentous fungal species Rhizomucor miehei and Rhizomucor pusillus and protease naturally produced by the fungal species Cryphonectria parasitica. Enzymes having milk clotting activity are also produced naturally by other fungal species including Rhizopus species, Physarum species and Penicillium species, and Bacillus species.

Generally, currently applied processes for manufacturing of commercial rennet products include steps of recovering the active enzymes from crude extracts or microbial fermentation media, followed by one or more purification steps.

As one example, WO 90/15866 discloses a method for recovering and purifying chymosin from an aqueous solution which includes the addition of polyethylene glycol and an inorganic salt so as to form a two phase system whereby the chymosin is concentrated in the PEG phase and cell debris and other impurities in the salt phase, followed by recovering the chymosin-rich phase and isolating the chymosin herefrom chromatographically. Such a multi-step process, however, is associated with several problems: (i) the process is labour intensive and the use of salts and PEG adds to the production cost, (ii) the use of PEG requires strict measures to be taken to remove this substance and (iii) the use of large amounts of salt represents a significant pollution problem. Furthermore, and importantly, the enzyme preparation obtained by eluting it from the chromatographic column may have an undesirably high content of enzymatic side activities requiring a further separation step, e.g. chromatography, to remove these undesired activities

Attempts have been made to develop a simpler process for recovery of milk clotting enzymes from crude aqueous media. Thus, WO 95/29999discloses the recovery of chymosin using a one-step chromatography process avoiding the use of PEG and salt.

However, such one-step processes frequently result in unsatisfactory yields of milk clotting enzyme, in particular when the conductivity of the crude preparation, such as a filtrate of a microbial fermentation medium, that is applied to the chromatographic column is high. Another very significant problem associated with such processes is that also in such a process, the eluate from the column may have undesired enzymatic side activities and thus a further chromatography step is required to remove such activities.

The present invention is based on the surprising discovery that undesired enzymatic activities in polypeptide preparations can be reduced or eliminated by a very simple process step of subjecting the preparation to low pH for an appropriate period of time without any significant concurrent inactivation of the active polypeptide contained in the preparation.

This achievement has made it possible to develop an efficient, cost-effective and less polluting method of recovering and isolating active polypeptides in a one-step chromatographic process from aqueous media in the form of preparations not containing undesired levels of enzymatic side activities

### SUMMARY OF THE INVENTION

Accordingly, in one aspect, the invention pertains to a method of providing a polypeptide preparation having a reduced content of undesired enzymatic side activities, the method comprising the steps of:

(i) providing a medium having a pH of 2.0 or higher that comprises at least one desired polypeptide and in addition hereto at least one undesired enzymatic side activity, and

(ii) subjecting said medium to a pH of less than 2.0 for a period of time that is sufficient to at least partially inactivate the at least one enzymatic side activity.

In a further aspect there is provided a milk clotting composition comprising an aspartic protease provided by the method of the invention, said composition essentially not having undesired enzymatic side activities.

### DETAILED DISCLOSURE OF THE INVENTION

It is a major objective of the present invention to provide a method whereby a preparation containing a biologically active polypeptide having a reduced content of undesired enzymatic side activities can be obtained whilst at the same time substantially retaining the biological activity of the polypeptide.

In the present context, the term “preparation containing a biologically active polypeptide” refers to any preparation containing a desired polypeptide which is manufactured and sold for a particular intended purpose, the term “polypeptide” encompasses peptides of two or more amino acids, i.e. the term encompasses molecules that are also generally referred to as peptides, oligopeptides or proteins. The term “biologically active” is meant to include any detectable activity that can be detected in in vitro systems and in vivo in both prokaryotic and eukaryotic organisms including higher organisms such as animals and plants. Thus, the term refers to e.g. antimicrobial, pharmaceutical, immunological (antibodies or antigens) or enzymatic activity. Additionally, as used herein the term “preparation” includes preparations in any form that is suitable for application or administration of the preparations, such as dry, semi-dry and liquid forms including e.g. suspensions, solutions or emulsions.

It is, as it is mentioned above, a common observation that preparations containing one or more desired polypeptides including raw materials and intermediate products used in the manufacturing of final products containing the polypeptide has a content of undesired enzymatic activities, also referred to herein as side activities. In many instances, such side activities have a detrimental effect which is exerted upon application of the desired polypeptide. As one typical example hereof, preparations of enzymes produced by extraction from the tissues of higher organisms or produced by cultivation of microorganisms, frequently contain minor amounts of undesired enzymatic activity. E.g. when milk clotting enzymes of both animal and microbial origin are produced using either organisms naturally producing such an enzyme or using recombinant host microorganisms having an inserted gene expressing the milk clotting enzyme, the producing strains produce a range of such side activities as described in the following. These side activities may restrict the field of application for the milk clotting enzyme and it is therefore common to set standards for content of side activities in such products which should be met.

The term “undesired enzymatic side activity” as used herein refers to any enzymatic activity, the presence of which in a polypeptide preparation is undesired for any reason such as detrimental or toxic effects occurring upon application or administration of the polypeptide preparation. As examples of such effects can be mentioned degradation of valuable components in a food product and immunologically adverse effects upon administration of a pharmaceutically active polypeptide. Undesired enzymatic activities encompassed by the present invention include as examples protease activity, starch degrading activity, peptidase activity, lipase activity, cellulase activity, lactase activity, hemicellulase activity, glucoamylase activity and phosphatase activity.

In the manufacturing of commercial polypeptide preparations the enzymatic side activity standards may be met by subjecting the products or the starting materials herefor to extensive purification processes which, however, in addition to the costs involved frequently implies loss of yield of the desired polypeptide.

In accordance with the method of the invention, the level of undesired side activities is reduced or eliminated using a very simple approach, i.e. by subjecting a starting material, an intermediate material or the final product having a pH 2.0 or higher that comprises at least one desired polypeptide and in addition hereto at least one undesired enzymatic side activity to a treatment where the pH is adjusted to less than 2.0 for a period of time that is sufficient to at least partially inactivate the at least one enzymatic side activity.

This acid treatment can be carried out using any suitable organic or inorganic acid, the use of which is compatible with the desired polypeptide and the applications herefor. A non-limiting range of such acids include lactic acid, acetic acid, citric acid, HCl, H₃PO₄, and H₂SO₄.

In preferred embodiments, this treatment at low pH results in that at least 75% of the activity of at least one desired polypeptide is retained after subjecting the medium having a pH of 2.0 or more to a pH or less than 2.0. More preferably, at least 80% such as at least 85% and most preferably, at least 90%, e.g. at least 95% of the activity of the desired polypeptide is retained.

Preferably, the method of the invention is capable of destroying or inactivating at least 50% of the activity of at least one undesired enzymatic activity present in the polypeptide preparation. In certain instances a lower degree of inactivation may be fully acceptable if this lower degree of inactivation results in that the final product meets the standard specifications for the particular side activity/activities. More preferably, at least 60% of the side activity is in activated by treatment, such as at least 70%, 80% or at least 90% of the activity of the undesired enzymatic side activity.

It will be appreciated that any starting material or intermediate material that is applied in the manufacturing of a preparation containing a desired polypeptide as well as the final product as such can be subjected to the treatment at low pH. Typical examples of such materials that are treated in accordance with the invention include media derived from the cultivation of a microorganism that during its cultivation produces at least one desired polypeptide and at least one undesired enzymatic side activity as defined herein. In accordance with the invention such media to be treated include media derived from cultivation of animal cells, plant cells and microbial cells including cells of a bacterial species such as a gram negative bacterial species including E. coli and a gram positive species including a Bacillus species, a yeast species and a species of filamentous fungi. Thus, media which can be treated by the method of the invention include media derived from the cultivation of cells of a yeast species selected from Saccharomyces cerevisiae, a methylotrophic yeast species including Pichia pastoris and a Klyuveromyces species, and media from cultivation of species of filamentous fungi such as e.g. Aspergillus species, Cryphonectria species, Fusarium species, Rhizomucor species and Trichoderma species. The undesired enzymatic activities in such cultivation media are primarily enzymatic side activities that are produced naturally by the production strain for the desired polypeptide. However, in specific embodiments, the undesired side activity is derived from a fusion partner for the desired polypeptide.

In accordance with the invention, the medium as defined above is subjected to a pH in the range of 1.0 to 1.99 such as in the range of 1.5 to 1.99 or in the range of 1.7 to 1.99. In useful embodiments, the treatment is at a pH of about 1.8.

The low pH treatment according to the invention is made for a period of time that is required to activate the side activity/activities to a desired level and that does not inactivate the biological activity of the desired polypeptide unacceptably. Typically, however, the required treatment period is within the range of 0.1 minutes to 48 hours such as a range of 1 minute to 36 hours including the range of 10 minutes to 24 hours. Those of skill in the art will be able to readily determine, for a particular polypeptide preparation, the appropriate treatment period using standard methods for assaying enzymatic activities and biological activity of the desired polypeptide.

In one particularly interesting embodiment, the preparation being treated is a preparation containing aspartic protease activity. As mentioned above, the class of aspartic proteases includes industrially important milk clotting enzymes that are widely used in cheese manufacturing. Accordingly, media which are treated in accordance with the invention include media derived from the cultivation of a microorganism that during the cultivation produces an aspartic protease and at least one undesired enzymatic side activity such as a medium that is derived from the cultivation of a microorganism that naturally produces the aspartic protease or a medium that is derived from the cultivation of a recombinant microorganism that has an inserted gene expressing the aspartic protease.

Non-recombinant and recombinant microorganisms that are useful in the production of aspartic proteases include bacterial species and yeast species such as those mentioned above.

Species of filamentous fungi are also widely used for the production of milk clotting aspartic proteases and suitable filamentous fungi for that purpose include species of Aspergillus, e.g. Aspergillus oryzae, Aspergillus nidulans or Aspergillus niger include Aspergillus niger var. awamori. Additionally, strains of a Fusarium species, e.g. Fusarium oxysporum or of a Rhizomucor species such as Rhizomucor miehei or a Trichoderma species including Trichoderma reseei and strains of Cryphonectria species including Cryphonectria parasitica can be used to produce milk clotting enzymes. Accordingly, the method of the present invention is used for inactivating side activities in media derived from cultivation of such fungal species.

In particular embodiments, the desired polypeptide in the preparation obtained herein is a fusion protein having, in addition to the aspartic protease activity, at least one undesired enzymatic side activity such as an amylase or glucoamylase activity.

In accordance with the invention, the desired polypeptide in the preparation that is obtained is an aspartic protease derived from the group consisting of an animal aspartic protease including a mammalian aspartic protease including pro-chymosin, chymosin, pepsinogen and pepsin, a plant aspartic protease and a microbial aspartic protease. A mammalian aspartic protease can be derived from any mammal species such as a ruminant species including a bovine species, an ovine species, a caprine species, a deer species, a buffalo species, an antelope species and a giraffe species, a Camelidae species including Camelus dromedarius, a porcine species, an Equidae species and a primate species. As it is mentioned above, a commonly used milk clotting enzyme preparation is based on extracts of stomach tissues of mammals naturally producing a milk clotting aspartic protease. Accordingly, the method of the invention is applicable to such preparations, including preparations of aspartic proteases derived from a naturally produced aspartic protease by the addition or deletion of one or more amino acids or substituting one or more amino acids herein.

It is a further objective of the present invention to provide a milk clotting composition consisting of a preparation comprising an aspartic protease provided by the method of invention and which essentially does not have undesired enzymatic side activities including such activities selected from glucoamylase activity, lactase activity, starch degrading enzyme activity, protease activity, peptidase activity, phosphatase activity, lipase activity, cellulase activity and hemicellulase activity. Such compositions may, in addition to the active component contain one or more additives as conventionally used in the manufacturing of preparations of milk clotting enzymes (i.e. coagulants or rennets derived from animal or microbial sources.

The invention will now be further explained in the following, non-limiting examples and the drawings where:

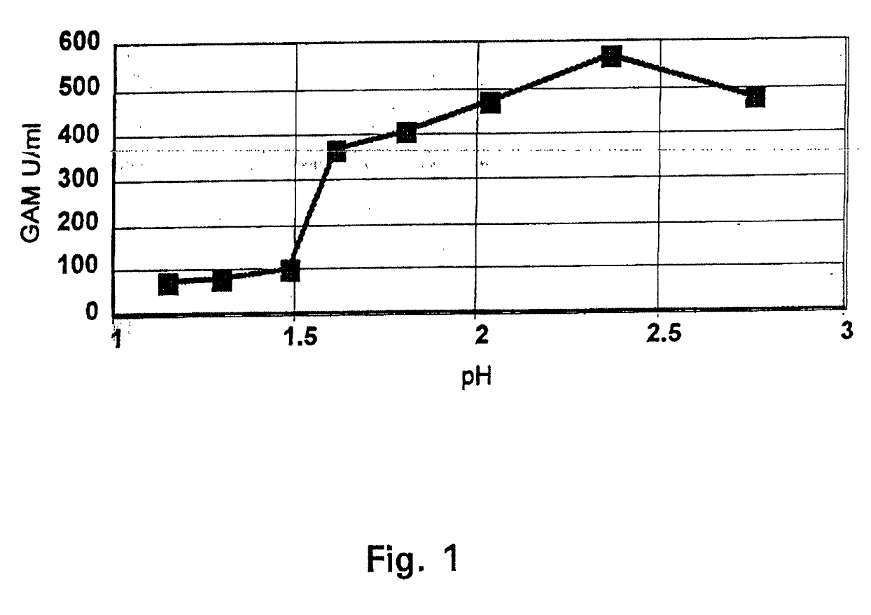

FIG. 1 illustrates the inactivation of glucoamylase activity in a fermentation medium for Aspergillus niger var. awamori expressing bovine chymosin at different pH values, and

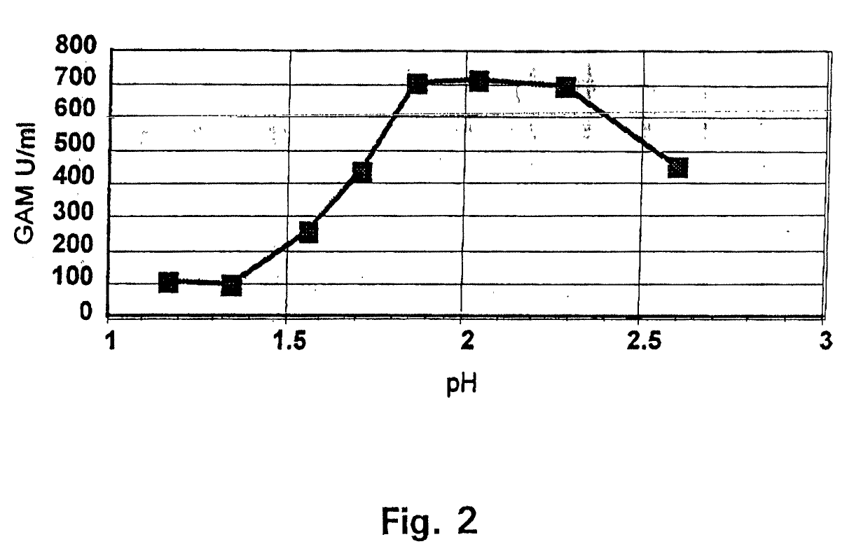

FIG. 2 shows the inactivation of Sigma glucoamylase at different pH values.

### EXAMPLE 1

Inactivation of Glucoamylase in a Recombinant Chymosin Preparation

In this study, a filtrate derived from the cultivation of a recombinant strain of Aspergillus niger var. awamori expressing a prochymosin-glucomylase fusion protein was used as test material. The filtrate was obtained from an industrial fermentation process by acidifying the cultivation medium after completion of the fermentation to inactivate the fungal biomass followed by separating the biomass by filtration.

The crude filtrate had a pH of about 2.2, contained 31.13 U/ml of glucoamylase activity and had a conductivity of 19 mS (1 GAM unit is defined as the amount of activity that hydrolyses starch by generating 1 μg glucose per min under the below standard conditions). The milk clotting activity of the filtrate was 98.5 International Milk Clotting Units (IMCUs) per ml. Prior to testing, the filtrate was diluted 5 times with distilled water. As a control, a commercial preparation of pure glucoamylase (GAM) derived from Aspergillus niger (Sigma No. A3514) was included as a solution containing 0.05 mg/ml distilled water.

Each of the above test solutions were adjusted to 8 different pH values within the range of about 1.1 to 2.7 (2.76, 2.36, 2.03, 1.80, 1.61, 1.48, 1.30 and 1.15, respectively) and incubated at room temperature for about 20 hours. Following this incubation, the GAM activity was measured using a solution of starch (e.g Merck Art 1257) prepared by dissolving 1 g in 100 ml 0.15 M acetate, pH 4.5 as substrate. For the assay, 500 μl of substrate solution, preheated in a water bath at 40° C., was mixed with 500 μl of enzyme solution to be tested and the mixture incubated for 20 minutes at 40° C. The reaction was stopped by the addition of 500 μl 0.3 M Tris. The amount of glucose formed was determined by mixing 200 μl of each of the reaction mixtures and 200 μl of a glucose standard containing 91 μl glucose/ml with 5.0 ml GOD-Perid reagent (Boehringer Mannheim 124028) reconstituted as recommended by the manufacturer. These mixtures were incubated at room temperature for 40 minutes followed by measuring E₆₁₀ using a spectrophotometer.

The results, which are summarised in FIG. 1 (filtrate) and 2 (Sigma GAM), demonstrate that it is possible to inactivate a substantial part of the GAM activity in the crude chymosin-containing preparation by subjecting the preparation to pH lower than 2.0.

### EXAMPLE 2

Inactivation of Enzymatic Side Activities in a Filtrate of Fermentation Medium of a Recombinant Chymosin-Producing Aspergillus niger var. awamori Strain

The starting material for this experiment was a fresh fermentate of Aspergillus niger var. awamori transformed with a plasmid expressing a prochymosin-glucoamylase fusion protein. The fermentate contained a milk clotting activity of 157.5 IMCU/ml, pH 5.59.

Samples to be tested in the experiment were prepared as follows: To 600 ml of the crude fermentate 5.4 ml of 100% acetic acid (0.9%) was added and pH was adjusted to 2.5 by addition of concentrated sulphuric acid (5 ml). A 50 ml sample was withdrawn and pH further adjusted to 1.8, 1.7 and 1.6, respectively, and a 50 ml sample collected at each pH. As control 50 ml of fermentate before pH adjustment was used.

Each of the above samples were centrifuged at 3.000 rpm for 10 minutes and subsequently filtered using a prefilter followed by filtration through a 0.45 μm filter. The samples were left to stand at 4° C. for about 21 hours and the pH of each sample adjusted to 5.5-6.0 to stop further reaction.

Following the acid treatment, the respective samples were assayed for residual milk clotting activity according to International IDF Standard 157A:1997 and for the following enzymatic side activities: GAM, leucine aminopeptidase (LAP), amylase, cellulase, acid phosphatase and protease activity, respectively. GAM activity was assayed as in Example 1.

The LAP activity was assayed using Leu-β-naphtylamine as a substrate, which, in the presence of LAP activity, release β-naphtylamine which is converted to a blue azo dye by first reacting it with NaNO₂ resulting in the generation of a diazo reagent which in a subsequent step is reacted with N-[1-naphtyl]-ethylenediamine to obtain the blue azo dye. In the assay, 125 μl of sample and 125 μl of LAP substrate is reacted for 1 hour at 37° C. and the reaction is terminated by the addition of 125 μl of 2 N HCl. The colour reaction is measured spectrophotometrically and the LAP activity determined vs. a standard curve. One peptidase unit is defined as the amount of enzyme releasing 1 μmol of β-naphtylamine from Leu-β-naphtylamide per hour at 37° C. and pH 7.1.

Amylase activity (1 unit defined as the amount of enzyme releasing 1 μmole of reducing groups calculated as maltose per minute under the below conditions) was assayed using soluble starch (Merck Art 1257) as the substrate at a concentration of 1% in 0.15 M sodium acetate, pH 4.8. Equal volumes of substrate and sample were mixed and incubated for 0 and 5 minutes, respectively at 25° C. followed by addition of an equal volume of a colour reagent (1 g 3.5-dinitrosalicylic acid is suspended in 20 ml 2 N NaOH and 50 ml distilled water is added followed by the addition of 30.0 g KNa tartrate and dilution to 100 ml) and boiling for 5 minutes followed by cooling in an ice bath. The enzyme activity was determined spectrophotometrically vs. a standard curve.

Cellulase activity was assayed using microcrystalline cellulose (Avicel-FMC Corp.) as substrate using a GOD-Perid reagent (Boehringer Mannheim 124028) including a 91 μg/ml glucose standard. For the assay, 100 mg substrate, 2.0 ml 0.15 M sodium acetate buffer, pH 5.0 and 40 μl 5% Na azide was mixed and 0.5 ml of the sample added. The reaction mixture is incubated for 24 hours at 37° C. followed by centrifugation. 200 μl of the resulting supernatant is mixed with 5.0 ml GOD-Perid reagents and the mixture kept at room temperature for 40 minutes and the colour development measured spectrophotometrically.

The assay or acid phosphatase is based on enzymatic hydrolysis of p-nitrophenyl phosphate. In the reaction, p-nitrophenol and inorganic phosphate are generated. When subjected to alkaline conditions, p-nitrophenol is converted to a yellow complex that can be measured at 400-420 nm. For the assay, a mixture of 0.5 ml substrate solution and 0.5 ml citrate buffer is prepared and 0.2 ml of sample is added followed by incubating the resulting mixture at 37° C. for 30 minutes. The reaction is stopped by the addition of NaOH and the absorbance at 420 nm determined after 10 minutes.

General protease activity was measured using azo casein as the substrate. One unit of protease activity is defined as the amount of enzyme that provides a ·E₄₂₅ of 1.00 per minute at 30° C. under defined conditions. For the assay, equal volumes of gel filtered sample at pH 5.2 and 5% azo casein at pH 6.7 is incubated in a 30° C. water bath for exactly 30 minutes after which the reaction is stopped by adding 1.5 ml 5% TCA while mixing. The reaction tube is cooled in ice bath and centrifuged until a clear supenatant is obtained. One ml of the supernatant is mixed with 2 ml 4 NaOH and the extinction measured spectophotometrially at 425 nm.

The contents of milk clotting activity in the samples of fermentate subjected to low pH for about 21 hours are summarised in the below table 2.1:

TABLE 2.1
Milk clotting activity in samples of chymosin-producing Aspergillus
niger var. awamori fermentate after 21 hours of low pH treatment

| | Milk clotting | Residual milk clot- |
|---|---|---|
| Sample | activity, IMCU/ml | ting activity, % |
| Filtrate of fermentate, pH 5.6 | 158 | 100 |
| Filtrate treated at pH 1.8 | 136 | 86.1 |
| Filtrate treated at pH 1.7 | 138 | 87.3 |
| Filtrate treated at pH 1.6 | 137 | 86.7 |

As it appears from the above table, more than 85% of the milk clotting activity of chymosin was retained even at pH 1.6.

In the following tables, the results for residual enzymatic side activities are summarised:

TABLE 2.2
GAM activity in samples of chymosin-producing Aspergillus niger
var. awamori fermentate after 21 hours of low pH treatment

| | | Residual GAM ac- |
|---|---|---|
| Sample | GAM activity, U/ml | tivity, % |
| Filtrate of fermentate, pH 5.6 | 30,619 | 100 |
| Filtrate treated at pH 1.8 | 4,020 | 13.1 |
| Fiitrate treated at pH 1.7 | 234 | 0.8 |
| Filtrate treated at pH 1.6 | 0 | 0 |

TABLE 2.3
Peptidase activity in samples of chymosin-producing Aspergillus niger
var. awamori fermentate after 21 hours of low pH treatment

| | Peptidase activity, | Residual peptidase |
|---|---|---|
| Sample | U/ml | activity, % |
| Filtrate of fermentate, pH 5.6 | 209 | 100 |
| Filtrate treated at pH 1.8 | 0.3 | 0.1 |
| Filtrate treated at pH 1.7 | 0 | 0 |
| Filtrate treated at pH 1.6 | 0 | 0 |

TABLE 2.4
Amylase activity in samples of chymosin-producing Aspergillus niger
var. awamori fermentate after 21 hours of low pH treatment

| | Amylase activity, | Residual amylase |
|---|---|---|
| Sample | U/ml | activity, % |
| Filtrate of fermentate, pH 5.6 | 34 | 100 |
| Filtrate treated at pH 1.8 | 0.11 | 0.05 |
| Filtrate treated at pH 1.7 | 0.04 | 0.01 |
| Filtrate treated at pH 1.6 | 0.02 | 0 |

TABLE 2.5
Cellulase activity in samples of chymosin-producing Aspergillus niger var.
awamori fermentate after 21 hours of low pH treatment

| | Peptidase activity, | Residual peptidase |
|---|---|---|
| Sample | U/ml | activity, % |
| Filtrate of fermentate, pH 5.6 | 170 | 100 |
| Filtrate treated at pH 1.8 | 17.6 | 10.3 |
| Filtrate treated at pH 1.7 | 15.0 | 8.8 |
| Filtrate treated at pH 1.6 | 28.7 | 16.9 |

TABLE 2.6
Phosphatase activity in samples of chymosin-producing Aspergillus niger
var. awamori fermentate after 21 hours of low pH treatment

| | Phosphatase activ- | Residual phospha- |
|---|---|---|
| Sample | ity, U/ml | tase activity, % |
| Filtrate of fermentate, pH 5.6 | 706 | 100 |
| Filtrate treated at pH 1.8 | 397 | 56.2 |
| Filtrate treated at pH 1.7 | 401 | 56.8 |
| Filtrate treated at pH 1.6 | 372 | 52.7 |

TABLE 2.7
Protease activity in samples of chymosin-producing Aspergillus niger var.
awamori fermentate after 21 hours of low pH treatment

| | Protease activity, | Residual protease |
|---|---|---|
| Sample | U/ml | activity, % |
| Filtrate of fermentate, pH 5.6 | 2 | 100 |
| Filtrate treated at pH 1.8 | 1 | 50 |
| Filtrate treated at pH 1.7 | 0 | — |
| Filtrate treated at pH 1.6 | 1 | 50 |

It appears from the above tables 2.2 to 2.7 that, with the exception of acid phosphatase activity, all the side activities being assayed were reduced by 50% or more by subjecting the fermentate to a 21 hours of treatment at pH values below 2.0. At the same time, more than 85% of the milk clotting activity of chymosin was retained during that treatment.

### EXAMPLE 3

Inactivation of Enzymatic Side Activities in Final Ready-to-Use Rennet Products by Low pH Treatment

In this experiment, samples of the following commercial microbial and animal rennet products were subjected to pH 1.7 for 2.5 hours and the GAM activity and the overall starch degrading activity was measured before and after that treatment.

The tested products were: Hannilase™195, a microbial coagulant produced by Rhizomucor miehei, Hannilase™2100, also a microbial rennet produced by Rhizomucor miehei, CHY-MAX™, a bovine chymosin produced by Aspergillus niger var. awamori, Modilase™ 195, a oxidised, thermobile coagulant derived from Rhizomucor miehei and Thermolase™, a microbial coagulant produced by Cryphonectria parasitica.

The above products were tested for GAM activity using the assay described in Example 1 and for total starch degrading activity using an agar diffusion test. The results of this assay was indicated as +++, ++, + or (+), where (+) indicates trace activity only observed after extended incubation.

The results of this experiment are summarised in the below tables:

TABLE 3.1
GAM activity in samples of commercial rennet products before and after low pH treatment

| | GAM activity, U/ml | GAM activity, U/ml | |
|---|---|---|---|
| | before low pH | after low pH | |
| Rennet product | treatment | treatment | Residual GAM activity, % |
| Hannilase ™ 195 | 212 | 1.4 | 0.7 |
| Hannilase ™ 2100 | 1254 | 2.1 | 0.2 |
| CHY-MAX ™ | 14 | 1.7 | 12.1 |
| Modilase ™ | 327 | 1.4 | 0.4 |
| Thermolase | 101 | 46 | 45.5 |

TABLE 3.2
General starch degrading activity in samples of commercial rennet
products before and after low pH treatment

| | Amylase activity | Amylase | Residual |
|---|---|---|---|
| | before low pH | activity after | amylase |
| Rennet product | treatment | low pH treatment | activity, % |
| Hannilase ™ 195 | +++ | (+) | 0 |
| Hannilase ™ 2100 | +++ | (+) | 0 |
| CHY-MAX ™ | not detectable | not detectable | 0 |
| Modilase ™ | +++ | (+) | 0 |
| Thermolase | +++ | (+) | 0 |

It can be concluded from the above results that subjecting commercial rennet products of microbial and animal origin to a treatment at pH less than 2.0 can reduce the level of GAM activity significantly and in any case by more than 50%, and the treatment inactivates the total starch degrading activity to levels which are close to being undetectable by the used diffusion assay.

1. A method of providing a polypeptide preparation having a reduced content of undesired enzymatic side activities, the method comprising the steps of:
(i) providing a medium having a pH of 2.0 or higher that comprises at least one desired polypeptide and in addition hereto at least one undesired enzymatic side activity, and
(ii) subjecting said medium to a pH of less than 2.0 for a period of time that is sufficient to at least partially inactivate the at least one enzymatic side activity.

2. A method according to claim 1 wherein at least 75% of the activity of the at least one desired polypeptide is retained after subjecting the medium having a pH of 2.0 or more to a pH of less than 2.0.

3. A method according to claim 2 wherein at least 85% of the activity of the at least one desired polypeptide is retained.

4. A method according to any of claims 1-3 wherein at least 50% of the activity of the at least one undesired enzymatic activity is inactivated.

5. A method according to claim 4 wherein at least 90% of the activity of the at least one undesired enzymatic activity is inactivated.

6. A method according to any of claims 1-5 wherein the medium having a pH of 2.0 or higher is a medium derived from the cultivation of an organism that during its cultivation produces the at least one desired polypeptide and the at least one undesired enzymatic side activity.

7. A method according to any of claims 1-6 wherein the at least one desired polypeptide is selected from the group consisting of an enzyme, an antibody, an antigen and a pharmaceutically active polypeptide.

8. A method according to any of claims 1-7 wherein the at least one enzymatic side activity is selected from the group consisting of glucoamylase activity, starch degrading enzyme activity, protease activity, peptidase activity, phosphatase activity, lipase activity, cellulase activity, lactase activity and hemicellulase activity.

9. A method according to any of claims 1-8 wherein the medium having a pH of 2.0 or higher is derived from the cultivation of an organism that is selected from the group consisting of an animal species, a plant species, a bacterial species, a yeast species and a species of filamentous fungi.

10. A method according to claim 9 wherein the bacterial species is selected from the group consisting of a gram negative bacterial species including E. coli and a gram positive species including a Bacillus species.

11. A method according to claim 9 wherein the yeast species is selected from the group consisting of Saccharomyces cerevisiae, a methylotrophic yeast species including Pichia pastoris and a Klyuveromyces species including Klyuveromyces lactis.

12. A method according to claim 9 wherein the species of filamentous fungi is selected from the group consisting of an Aspergillus species, a Cryphonectria species, a Fusarium species, a Rhizomucor species and a Trichoderma species.

13. A method according to any of claims 1-12 wherein the medium having a pH of 2.0 or higher is subjected to a pH in the range of 1.0 to 1.99.

14. A method according to claim 13 wherein the pH is in the range of 1.5 to 1.99.

15. A method according to claim 14 wherein the pH is in the range of 1.7 to 1.99.

16. A method according to claim 15 wherein the pH is about 1.8.

17. A method according to any of claims 13-16 wherein the pH in the range of 1.0 to 1.99 is provided by adding an inorganic or an organic acid.

18. A method according to any of claims 1-17 wherein the medium having a pH of 2.0 or higher is subjected to a pH of less than 2.0 for a period of time that is in the range of 0.1 minutes to 48 hours.

19. A method according to any of claims 1-18 wherein the at least one desired polypeptide has aspartic protease activity.

20. A method according to claim 19 wherein the medium having a pH of 2.0 or higher is a medium derived from the cultivation of a microorganism that during the cultivation produces the aspartic protease and the at least one undesired enzymatic side activity.

21. A method according to claim 20 wherein the medium is derived from the cultivation of a microorganism that naturally produces the aspartic protease or from the cultivation of a recombinant microorganism that has an inserted gene expressing the aspartic protease.

22. A method according to claim 21 wherein the microorganism is selected from the group consisting of a bacterial species, a yeast species and a species of filamentous fungi.

23. A method according to claim 22 wherein the aspartic protease is expressed as a fusion protein having, in addition to the aspartic protease activity, at least one undesired enzymatic side activity.

24. A method according to claim 23 wherein the at least one enzymatic side activity is starch degrading enzyme activity including an activity selected from the group consisting of amylase activity and glucoamylase activity.

25. A method according to any of claims 20-24 wherein the microorganism is one that naturally produces at least one enzymatic side activity.

26. A method according to claim 25 wherein the at least one enzymatic side activity is selected from the group consisting of glucoamylase activity, lactase activity, starch degrading enzyme activity, protease activity, peptidase activity, phosphatase activity, lipase activity, cellulase activity and hemicellulase activity.

27. A method according to any of claims 19-26 wherein the aspartic protease is derived from the group consisting of an animal aspartic protease including a mammalian aspartic protease, a plant aspartic protease and a microbial aspartic protease.

28. A method according to claim 27 wherein the mammalian aspartic protease is selected from the group consisting of pro-chymosin, chymosin, pepsinogen and pepsin.

29. A method according to claim 28 wherein the aspartic protease is derived from a mammalian species selected from the group consisting of a ruminant species, a Camelidae species including Camelus dromedarius, a porcine species, an Equidae species and a primate species.

30. A method according to claim 29 wherein the ruminant species is selected from the group consisting of a bovine species, an ovine species, a caprine species, a deer species, a buffalo species, an antelope species and a giraffe species.

31. A method according to any of claims 27-30 wherein the mammalian derived aspartic protease is a protease naturally produced in a mammalian species.

32. A method according to claim 27 wherein the aspartic protease is derived from a naturally produced aspartic protease by the addition or deletion of one or more amino acids or substitution of one or more amino acids herein.

33. A milk clotting composition comprising a preparation of an aspartic protease, provided by the method of any of claims 1-32, said composition essentially not having undesired enzymatic side activities.

34. A method according to claim 33 essentially not having an undesired enzymatic side activity selected from the group consisting of glucoamylase activity, lactase activity, starch degrading enzyme activity, protease activity, peptidase activity, phosphatase activity, lipase activity, cellulase activity and hemicellulase activity.
